(19) United States
(12) Patent Application Publication
(10) Pub. No.: US 2025/0136911 A1
(43) Pub. Date: May 1, 2025

(54) CELL CULTURE MEMBRANE AND MEASUREMENT METHOD

(71) Applicants: TOYODA GOSEI CO., LTD., Kiyosu-shi (JP); TOHOKU UNIVERSITY, Sendai-shi (JP)

(72) Inventors: Sayaka MIYATA, Aichi (JP); Yasuhiko SHINODA, Aichi (JP); Seitaro TAKI, Aichi (JP); Kosuke INO, Miyagi (JP); Yoshinobu UTAGAWA, Miyagi (JP); Hitoshi SHIKU, Miyagi (JP)

(21) Appl. No.: 18/928,894

(22) Filed: Oct. 28, 2024

(30) Foreign Application Priority Data
Oct. 30, 2023 (JP) 2023-185739

(51) Int. Cl. C12M 1/12; C12M 1/00; C12M 1/34

(52) U.S. Cl. CPC C12M 25/02; C12M 23/20; C12M 41/26

(57) ABSTRACT

A cell culture membrane includes: a membrane body having a first face and a second face located on a side opposite to the first face; a metal film including at least one of a first metal film provided to overlap the first face or a second metal film provided to overlap the second face; and a polyaniline film provided to overlap the metal film, and the membrane body has a plurality of holes, and each of the plurality of holes has a mortar shape and opens at least at the first face.

### CROSS-REFERENCE TO RELATED APPLICATION

This application is based on and claims priority under 35 USC 119 from Japanese Patent Application No. 2023-185739 filed on Oct. 30, 2023.

### TECHNICAL FIELD

The present disclosure relates to a cell culture membrane and a measurement method.

### BACKGROUND ART

Conventionally, in order to evaluate the state of cell culture, measurement of a physical property value of a culture solution and measurement of the amount of a specific substance in the culture solution are performed (for example, JP2004-113092A). JP2004-113092A describes a cell culture chip provided with a pH sensor that measures the physical property value of a culture solution. In this cell culture chip, the pH sensor is disposed above a well in which cells are cultured.

### SUMMARY OF INVENTION

In the above cell culture chip, since cells are cultured on a bottom face of the well, there may be cases where pH measured by the pH sensor does not reflect pH in the vicinity of the cells.

The present disclosure can be implemented by the following aspects.
(1) According to an aspect of the present disclosure, a cell culture membrane is provided. The cell culture membrane includes: a membrane body having a first face and a second face located on a side opposite to the first face; a metal film including at least one of a first metal film formed to overlap the first face or a second metal film formed to overlap the second face; and a polyaniline film formed to overlap the metal film, in which the membrane body has a plurality of holes, and each of the plurality of holes has a mortar shape and opens at least at the first face. According to this aspect, in a case where cells are cultured on the polyaniline film, since the polyaniline film is in contact with or close to the cells, pH in the vicinity of the cells can be accurately measured by using the metal film as an electrode and using the polyaniline film as a response film of protons.
(2) In the cell culture membrane according to the above aspect, each of the plurality of holes may be a non-through hole that does not open at the second face and has an inner peripheral portion that defines the each of the plurality of holes, the metal film may not include the second metal film and may include the first metal film, the first metal film may have a metal inner peripheral portion covering the inner peripheral portion, and the polyaniline film may have a polyaniline inner peripheral portion covering the metal inner peripheral portion. According to this aspect, since it is possible to increase an area of the polyaniline film in contact with or close to the cells that have entered the holes, measurement accuracy of the pH in the vicinity of the cells can be further improved.
(3) In the cell culture membrane according to the above aspect, each of the plurality of holes may be a through hole penetrating from the first face to the second face, the plurality of holes may include a first opening portion opening at the first face, a second opening portion opening at the second face, and an inner peripheral portion connecting the first opening portion and the second opening portion, the metal film may include the first metal film without including the second metal film, the first metal film may include a metal inner peripheral portion formed to cover the inner peripheral portion, and the polyaniline film may have a polyaniline inner peripheral portion formed to cover the metal inner peripheral portion. According to this aspect, when different types of cells are cultured on the first face and the second face, respectively, since the cells cultured on the first face and the cells cultured on the second face can come into contact with each other through the through holes, a cellular condition closer to that in a living body can be created.
(4) In the cell culture membrane according to the above aspect, each of the plurality of holes may be a through hole penetrating from the first face to the second face, and have a first opening portion opening at the first face, a second opening portion opening at the second face, and an inner peripheral portion connecting the first opening portion to the second opening portion, the metal film may not include the second metal film and include the first metal film, the first metal film may have a metal inner peripheral portion formed to cover the inner peripheral portion and a metal closing portion closing the second opening portion, the polyaniline film may have a polyaniline inner peripheral portion formed to cover the metal inner peripheral portion and the metal closing portion, and the metal closing portion may have a crack. According to this aspect, the cells cultured on the first face and the cells cultured on the second face can come into contact with each other through the crack, and since the size of the crack is smaller than the size of the second opening portion, it is expected that the cellular condition closer to that in the living body can be created.
(5) In the cell culture membrane according to the above aspect, the plurality of holes may have a first average hole diameter at the first face larger than a second average hole diameter at the second face. According to this aspect, it is possible to increase the possibility that the cells can be cultured in a state in which the cells enter the holes from the first opening portion and enter the holes without passing through the second opening portion.
(6) In the cell culture membrane according to the above aspect, the membrane body may be formed of polyurethane. According to this aspect, it is possible to easily produce the membrane body having the plurality of through holes whose first average hole diameter is larger than the second average hole diameter at the second face.
(7) In the cell culture membrane according to the above aspect, the second average hole diameter may be 7 um or less. According to this aspect, when the cells having a size of about 10 um are cultured, it is possible to prevent the cells cultured on the first face or the second face from moving to the other of the first face and the second face through the through holes.
(8) In the cell culture membrane according to the above aspect, the metal film may include a first metal layer mainly made of gold (Au). According to this aspect, the first metal layer mainly made of gold can be used as the metal film.
(9) In the cell culture membrane according to the above aspect, the metal film may further include a second metal layer mainly made of platinum (Pt) and a third metal layer mainly made of titanium (Ti). According to this aspect, a stacked film including the first metal layer mainly made of gold, the second metal layer mainly made of platinum, and the third metal layer mainly made of titanium can be used as the metal film.
(10) A measurement method of pH using the cell culture membrane according to the above aspect, may include: a first step of immersing the cell culture membrane in a culture solution to culture a cell on the polyaniline film of the cell culture membrane; a second step of inserting a reference electrode into the culture solution, using the metal film as a working electrode, and measuring a potential difference between the reference electrode and the working electrode; and a third step of determining pH of the culture solution using the measured potential difference. According to this aspect, when the cells are cultured on the cell culture membrane, it is possible to measure the pH using the metal film as an electrode.

### BRIEF DESCRIPTION OF DRAWINGS

FIG. 1 is a schematic diagram of a cross section of a cell culture membrane.

FIG. 2 is a flowchart showing a manufacturing process of a cell culture membrane.

FIG. 3 is a flowchart showing a procedure of a measurement method of measuring pH.

FIG. 4 is a view showing a measurement system assembled in the case of measuring the pH.

FIG. 5 is a diagram illustrating an oxidation-reduction reaction of conductive polyaniline.

FIG. 6 is a graph showing a relationship between pH and a potential difference.

FIG. 7 is a schematic diagram in a case of co-culturing using the cell culture membrane.

FIG. 8 shows views of embodiments (C1) to (C3) of the cell culture membrane.

FIG. 9 shows views of embodiments (C4) to (C6) of the cell culture membrane.

FIG. 10 shows views of embodiments (C7) and (C8) of the cell culture membrane.

FIG. 11 is a view showing another embodiment of a culture container.

FIG. 12 shows SEM images obtained by observing membranes produced in steps of the manufacturing process of the cell culture membrane.

FIG. 13 shows SEM images obtained by observing the cell culture membrane on which cells are cultured.

FIG. 14 is a diagram illustrating a chemical reaction when ammonium chloride is added to an aqueous solution containing the cells.

FIG. 15 shows measurement results of open circuit potential when ammonium chloride was added to PBS.

FIG. 16 is an SEM image obtained by observing the cell culture membrane on which the cells are cultured.

### DESCRIPTION OF EMBODIMENTS

### A. Embodiments

### A-1. Configuration of Cell Culture Membrane

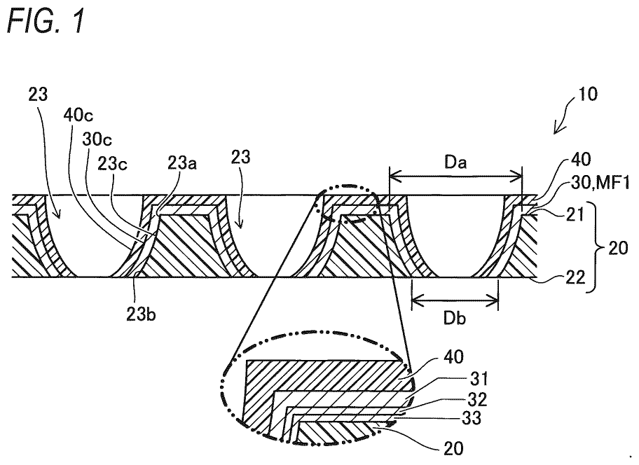

FIG. 1 is a schematic diagram of a cross section of a cell culture membrane 10. The cell culture membrane 10 includes a membrane body 20, a metal film 30, and a polyaniline film 40. In the present embodiment, the cell culture membrane 10 is formed of polyurethane. The metal film 30 is implemented by stacking a first metal layer 31 mainly made of gold (Au), a second metal layer 32 mainly made of platinum (Pt), and a third metal layer 33 mainly made of titanium (Ti) in this order from an upper layer toward a lower layer. Since the third metal layer 33 is disposed between the second metal layer 32 and the membrane body 20, adhesion of the first metal layer 31 and the second metal layer 32 to the membrane body 20 can be improved. As another embodiment of the metal film 30, the metal film 30 may include only the first metal layer 31 mainly made of gold (Au). Since the cell culture membrane 10 includes the metal film 30 and the polyaniline film 40, after cells are cultured on the polyaniline film 40, pH of the culture solution 305 to be described later can be measured by using the metal film 30 as an electrode and the polyaniline film 40 as a response film of protons.

The cell culture membrane 10 includes a membrane body 20 having a first face 21 and a second face 22 located on a side opposite to the first face 21, and a plurality of through holes 23 serving as a plurality of holes formed in the membrane body 20. The plurality of through holes 23 are holes penetrating from the first face 21 to the second face 22. Each through hole 23 has a first opening portion 23a opening at the first face 21, a second opening portion 23b opening at the second face 22, and an inner peripheral portion 23c connecting the first opening portion 23a to the second opening portion 23b. Since the cell culture membrane 10 has the second opening portions 23b, different types of cells can be brought into contact with each other when they are cultured on the first face 21 and the second face 22, respectively.

Each through hole 23 has a mortar shape. A hole diameter of the through hole 23 at the first face 21 is different from a hole diameter of the through hole 23 at the second face 22. The hole diameter of the through hole 23 at the first face 21 is referred to as a first hole diameter Da, and the hole diameter of the through hole 23 at the second face 22 is referred to as a second hole diameter Db. The first hole diameter Da is larger than the second hole diameter Db. In the case of the through hole, a shape in which the second hole diameter Db is smaller than the first hole diameter Da is encompassed by the mortar shape. Further, both a case where a shape of the inner peripheral portion 23c is a curved face and a case where the inner peripheral portion 23c is a plane are encompassed by the mortar shape. A first average hole diameter, which is an average value of the first hole diameters Da of the plurality of through holes 23 at the first face 21, is different from a second average hole diameter, which is an average value of the second hole diameters Db at the second face 22. The first average hole diameter is larger than the second average hole diameter. As will be described later, a cell 300 can be cultured in a state in which the cell 300 has entered the interior of the through hole 23. The second average hole diameter is preferably 7 μm or less. Accordingly, as will be described later, when co-culture is performed using the cell culture membrane 10, after a first cell 301 is seeded on the cell culture membrane 10, the first cell 301 can be prevented from moving to a face opposite to the seeded face after passing through the through hole 23. In the following description, the movement of the first cell 301 to the face opposite to the seeded face after passing through the through hole 23 may be referred to as “wrap-around”.

The first average hole diameter is a value obtained by irradiating the upward-facing first face 21 with light and performing observation with a microscope in a state in which the first face 21 is on the top and the second face 22 is on the bottom. Since the light is not reflected from the through hole 23, the through hole 23 is visually recognized in black. Specifically, circle equivalent diameters of all the through holes 23 observed in a specific visual field are measured and an average value of the measured values is obtained. The circle equivalent diameter refers to a diameter of a true circle corresponding to an area of the through hole 23. The second average hole diameter is similarly obtained. That is, the value is obtained by irradiating the upward-facing second face 22 with light and performing observation with a microscope in a state in which the second face 22 is on the top and the first face 21 is on the bottom.

The metal film 30 includes a first metal film MF1 formed to overlap the first face 21. The first metal film MF1 has a metal inner peripheral portion 30c. The metal inner peripheral portion 30c is formed to cover the inner peripheral portion 23c.

The polyaniline film 40 has a polyaniline inner peripheral portion 40c. The polyaniline inner peripheral portion 40c is formed to cover the metal inner peripheral portion 30c. Since the metal film 30 and the polyaniline film 40 are formed to cover the inner peripheral portion 23c, as will be described later, it is possible to further improve measurement accuracy of the pH in the vicinity of the cell 300.

### A-2. Manufacturing Method of Cell Culture Membrane

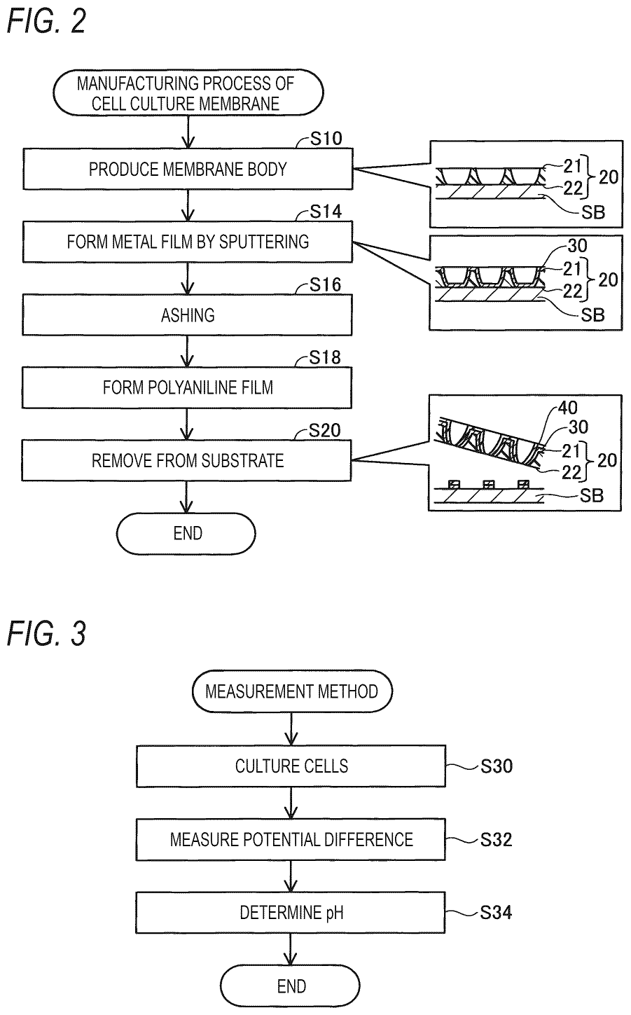

FIG. 2 is a flowchart showing a manufacturing process of the cell culture membrane 10. As shown in FIG. 2, in step S10, the membrane body 20 formed of polyurethane is produced. Specifically, water vapor is supplied to an uncured polyurethane raw material formed in a thin plate shape on a substrate SB, and the uncured polyurethane raw material is cured while foaming. According to this manufacturing method, since the through hole 23 is formed by foaming, the cell culture membrane 10 in which the first hole diameter Da and the second hole diameter Db are different from each other can be easily manufactured. Further, the cell culture membrane can be manufactured, for example, as described in JP6343492B.

In step S14, the metal film 30 is formed on the first face 21 of the membrane body 20 by sputtering. Specifically, the membrane body 20 formed on the substrate SB is placed in a vacuum chamber together with the substrate SB, and a metal is deposited on the first face 21 to form the metal film 30. In the present embodiment, sputtering time of the first metal layer 31 is three minutes. Sputtering time of the second metal layer 32 is one minute. Sputtering time of the third metal layer 33 is one minute. The sputtering time is not limited to the above.

In step S16, the metal film 30 is ashed. Specifically, in step S16, a surface of the metal film 30 is irradiated with oxygen plasma. Accordingly, it is possible to impart hydrophilicity to the surface of the metal film 30.

In step S18, the polyaniline film 40 is formed on the metal film 30. Specifically, the polyaniline film 40 is formed by electrolytic polymerization of aniline on the metal film 30. Accordingly, the polyaniline film 40 having conductivity is formed. Although the polyaniline film 40 is formed using a potential scanning method, the polyaniline film 40 may be formed using a constant potential method or a constant current method. In step S16, which is performed before step S18, the inner peripheral portion 23c of the through hole 23 can be modified with polyaniline because the metal film 30 is made hydrophilic.

In step S20, the membrane body 20 is removed from the substrate SB, and the manufacturing process ends. In the process of removing the membrane body 20 from the substrate SB, the metal film and the polyaniline film formed on the substrate SB are separated from the membrane body 20. Therefore, the second opening portion 23b of the membrane body 20 is not closed by the metal film.

### A-3. Measurement Method of pH

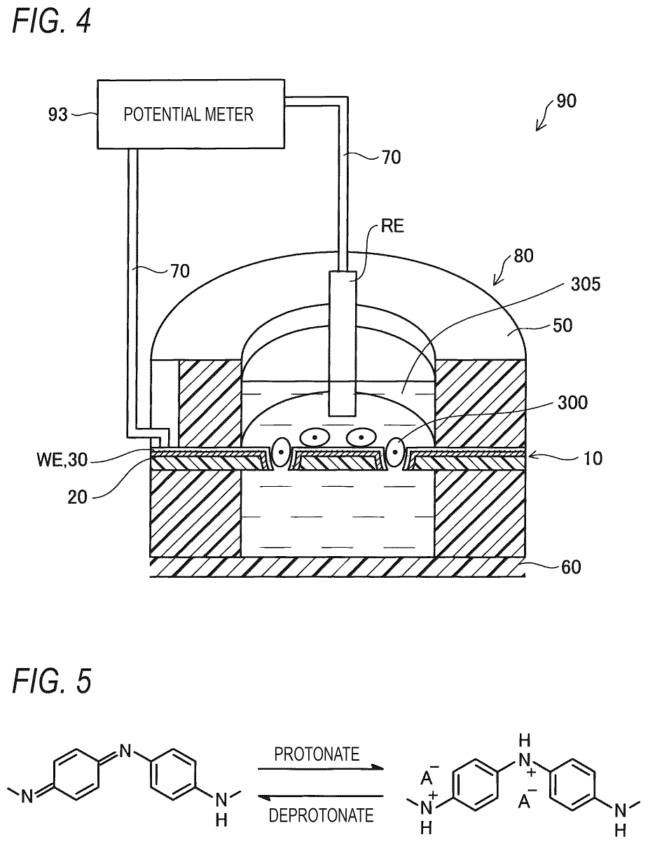

FIG. 3 is a flowchart showing a procedure of a measurement method of measuring pH of the culture solution 305 after the cells are cultured on the cell culture membrane 10. FIG. 4 is a view showing a measurement system 90 assembled in the case of measuring the pH.

As shown in FIG. 3, in the culture step of step S30, the cells 300 are cultured on the cell culture membrane 10. In the present embodiment, culture is performed using a culture container 80 including the cell culture membrane 10.

As shown in FIG. 4, the culture container 80 includes a cell culture membrane 10 and a cylindrical member 50. The cylindrical member 50 is made of a resin and has a cylindrical shape. The cell culture membrane 10 is attached to a central position in an axial direction of the cylindrical member 50. Thus, the culture container 80 functions as a cylindrical container having the cell culture membrane 10 as the bottom face. A conductive wire 70 is electrically connected to the metal film 30 of the cell culture membrane 10. In the present embodiment, a thickness of the cell culture membrane 10 is 5 μm. In the present embodiment, an inner diameter of the cylindrical member 50 is 8 mm.

In step S30 as a first step, in a state in which the culture container 80 is placed in the resin container 60, and the culture solution 305 is placed inside the cylindrical member 50, and the cells 300 are seeded and cultured.

In step S32 as a second step in FIG. 3, a potential difference is measured. As shown in FIG. 4, the measurement system 90 includes a potential meter 93 and a reference electrode RE. The conductive wire 70 electrically connected to the metal film 30 is electrically connected to the potential meter 93. In the measurement, the metal film 30 functions as a working electrode WE. The reference electrode RE is electrically connected to the potential meter 93 via a conductive wire 70. Specifically, in step S32, the reference electrode RE is inserted into the culture solution 305, and the potential difference between the metal film 30 functioning as the working electrode WE and the reference electrode RE is measured by the potential meter 93.

In step S34 following a third step of FIG. 3, the pH of the culture solution 305 is determined using the measured potential difference. Specifically, the pH is determined using a relational expression between the pH and the potential difference, which is determined in advance. Since the cell 300 is in contact with or close to the polyaniline film 40 and the metal film 30, the pH in the vicinity of the cell 300 can be accurately measured. Specifically, an approximate expression can be used as a relational expression between the pH and the potential difference. Further, the pH may be determined using a map representing a relationship between a range of the potential difference and the pH applied to each range.

### A-4. Measurement Principle of pH

FIG. 5 is a diagram illustrating an oxidation-reduction reaction of conductive polyaniline. As shown in FIG. 5, in the polyaniline, addition and desorption of protons are reversibly carried out. Thus, potential of the electrode modified with the conductive polyaniline changes depending on the pH.

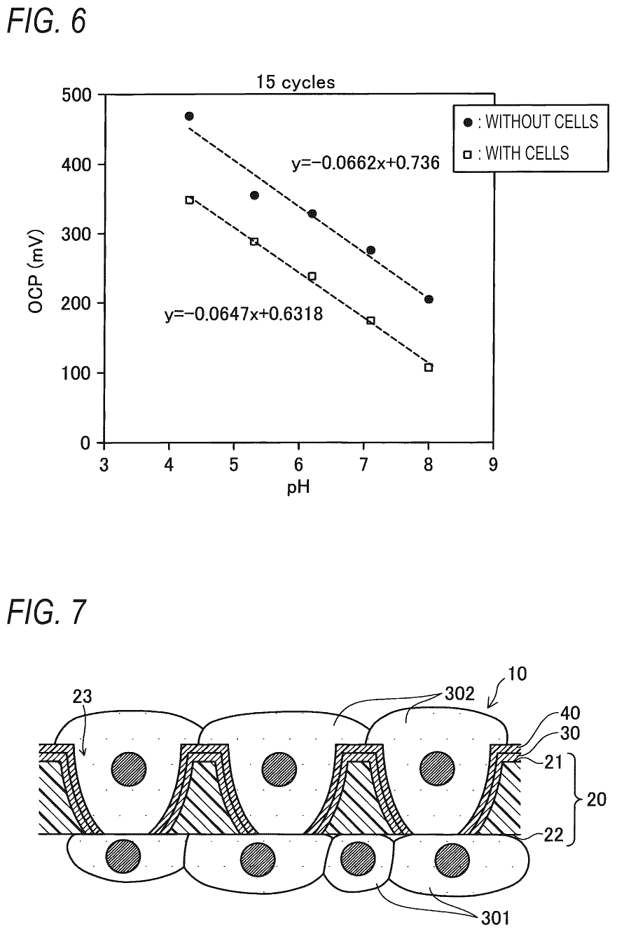

FIG. 6 is a graph showing the relationship between the pH and the potential difference. FIG. 6 shows an experimental result measured by inventors using the cell culture membrane 10. The cell culture membrane 10 was placed in a buffer solution having known pH, and an open circuit potential (OCP), which is a potential difference between the metal film 30 functioning as the working electrode WE and the reference electrode RE, was measured. A relationship between the pH and the open circuit potential was determined by measuring the potential difference for buffer solutions of various pH values. An acetic acid buffer solution was used as a buffer solution having pH of 4 to 5. A phosphate buffer solution was used as a buffer solution having pH of 6 to 8. The polyaniline film 40 is produced by a potential scanning method, and the number of sweeps is 15. The measurement was performed for each of the cell culture membrane 10 not subjected to cell culture and the cell culture membrane 10 subjected to cell culture. “Without cells” in FIG. 6 indicates the cell culture membrane 10 not subjected to the cell culture. “With cells” in FIG. 6 indicates the cell culture membrane 10 subjected to the cell culture.

It is assumed that the lower the pH of the buffer solution, the more the protonation of polyaniline progresses, and the higher the potential of the working electrode WE becomes, and thus the potential difference between the working electrode WE and the reference electrode RE increases. As shown in FIG. 6, it was confirmed that the open circuit potential is proportional to the pH, and the lower the pH, the higher an open circuit voltage. Accordingly, the pH of the culture solution 305 can be determined by obtaining the relational expression between the pH and the open circuit voltage in advance and measuring the open circuit voltage of the culture solution 305 whose pH is unknown using the cell culture membrane 10.

### B. Another Embodiment of Culture Method

The cell culture using the cell culture membrane 10 in step S30 of FIG. 3 is not limited to the case of cell culture on one face of the cell culture membrane 10, and may be cell culture on both faces of the cell culture membrane 10. In this case, co-culture of culturing two or more different types of cells on both faces of the cell culture membrane 10 may be performed.

FIG. 7 is a schematic diagram when first cells 301 and second cells 302 of a different type from the first cells 301 are co-cultured on the cell culture membrane 10. The cell culture membrane 10 is attached to, for example, an end portion of an insert accommodated in the well. In a first step of the co-culture using the cell culture membrane 10, the first cells 301 are seeded and cultured on the second face 22 in a state in which the second face 22 of the cell culture membrane 10 is oriented upward in a vertical direction. In the next second step, the second cells 302 are seeded and cultured on the metal film 30 in a state in which the first face 21 of the cell culture membrane 10 is oriented upward in the vertical direction. In the culture, the second cells 302 enter the through holes 23. Thus, the first cell 301 and the second cell 302 can come into contact with each other. For example, by using epithelial cells as the first cells 301 and using vascular endothelial cells as the second cells 302, a three-dimensional model closer to a living body can be produced.

The cell culture using the cell culture membrane 10 is not limited to the above. The same type of cells 300 may be cultured on the first face 21 and the second face 22, or two or more types of cells 300 may be cultured on each of the first face 21 and the second face 22.

### C. Other Embodiments of Cell Culture Membrane

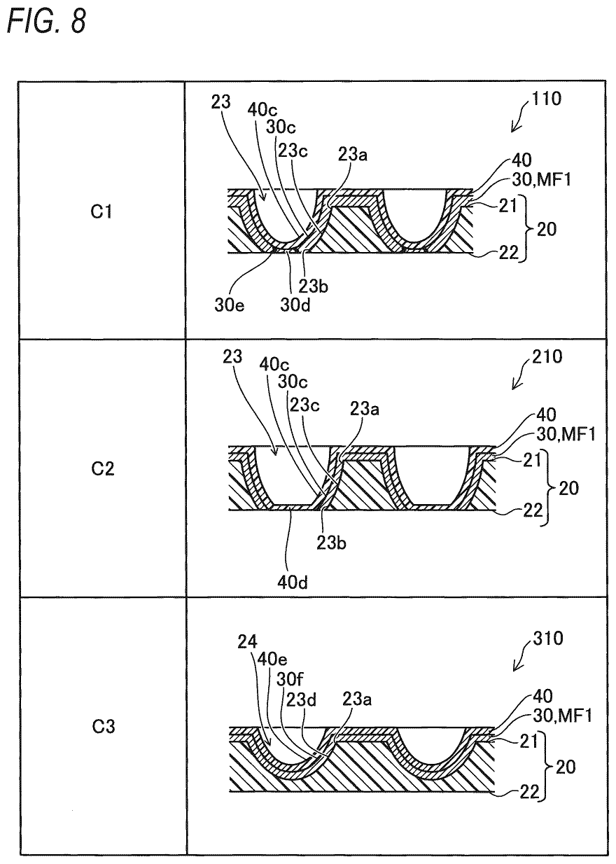

FIG. 8 shows views of embodiments (C1) to (C3), which are other embodiments, of the cell culture membrane 10. The same configurations as those of the above embodiment are denoted by the same reference numerals, and detailed description thereof will be appropriately omitted.

(C1) As shown in “C1” of FIG. 8, a cell culture membrane 110 includes the membrane body 20, the metal film 30, and the polyaniline film 40. The metal film 30 includes the first metal film MF1. The cell culture membrane 110 is different from the cell culture membrane 10 shown in FIG. 1 in that the first metal film MF1 includes metal closing portions 30d closing the second opening portions 23b of the membrane body 20. The metal closing portion 30d has a crack 30e. The polyaniline film 40 has the polyaniline inner peripheral portion 40c formed to cover the metal inner peripheral portion 30c and the metal closing portion 30d.

The cell culture membrane 110 is produced similarly to the manufacturing process shown in FIG. 2. However, in step S14, sputtering time is set to be longer than the sputtering time of the cell culture membrane 10. Specifically, sputtering time of the first metal layer 31 is nine minutes. Sputtering time of the second metal layer 32 is two minutes. Sputtering time of the third metal layer 33 is two minutes. Accordingly, a thickness of the metal film 30 of the cell culture membrane 110 is larger than a thickness of the metal film 30 of the cell culture membrane 10. Thus, when the membrane body 20 is removed from the substrate SB in step S20, the metal film 30 and the polyaniline film 40 formed on the substrate SB are not separated from the membrane body 20 and are maintained in a state of being coupled to the membrane body 20.

The crack 30e is a gap smaller than 3 um. Since the crack 30e is formed in the cell culture membrane 110, the first cell 301 and the second cell 302 can be brought into contact with each other via the crack 30e when the co-culture is performed. It is expected that the crack 30e can reproduce the role of a fine hole of the base membrane. Therefore, it is expected that a cellular condition closer to that in the living body can be recreated by culture using the cell culture membrane 110.

(C2) As shown in “C2” of FIG. 8, a cell culture membrane 210 includes the membrane body 20, the metal film 30, and the polyaniline film 40. The cell culture membrane 210 is different from the cell culture membrane 10 shown in FIG. 1 in that the polyaniline film 40 has metal closing portions 40d closing the second opening portions 23b. According to the cell culture membrane 210, since it is possible to increase an area of the polyaniline film 40 in contact with or close to the cells 300 that have entered the interior of the polyaniline inner peripheral portion 40c, the measurement accuracy of the pH in the vicinity of the cells 300 can be further improved.

(C3) As shown in “C3” of FIG. 8, a cell culture membrane 310 has the membrane body 20, the metal film 30, and the polyaniline film 40. The membrane body 20 is different from the cell culture membrane 10 shown in FIG. 1 in that the through holes 23 are not provided and a plurality of holes 24 that are non-through holes are provided. The hole 24 has a mortar shape. In the case of the non-through hole, the following shapes (A) and (B) are included in the mortar shape. The shape (A) refers to a shape in which an inner peripheral face defining the hole is substantially hemispherical with a vertex at a point where a distance between this inner peripheral face and the second face is the shortest. The shape (B) refers to a shape in which an area of a cross section of the inner peripheral face parallel to the first face at a position where the distance between the inner peripheral face defining the hole and the second face is shortest is smaller than an area of an opening portion of the hole at the first face. The holes 24 are defined by the inner peripheral portion 23d. The metal film 30 has a metal inner peripheral portion 30f that convers the inner peripheral portion 23d. The polyaniline film 40 has a polyaniline inner peripheral portion 40e covering the metal inner peripheral portion 30f. According to the cell culture membrane 310, since it is possible to increase an area of the polyaniline film 40 in contact with or close to the cells 300 that have entered the interior of the polyaniline inner peripheral portion 40e, the measurement accuracy of the pH in the vicinity of the cells 300 can be further improved.

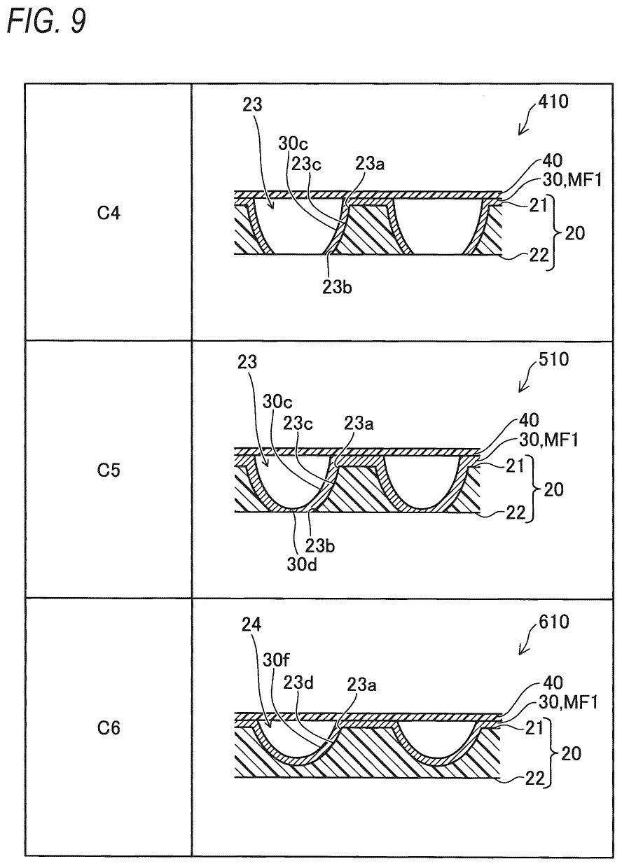

FIG. 9 shows views of embodiments (C4) to (C6), which are other embodiments, of the cell culture membrane 10. Common points of the embodiments (C4) to (C6) are that the polyaniline film 40 does not enter the through hole 23 or the hole 24. The same configurations as those of the above embodiment are denoted by the same reference numerals, and detailed description thereof will be appropriately omitted.

(C4) As shown in “C4” of FIG. 9, a cell culture membrane 410 includes the membrane body 20, the metal film 30, and the polyaniline film 40. Since the membrane body 20 and the metal film 30 have the same structure as those in the cell culture membrane 10 shown in FIG. 1, description thereof will be omitted. The polyaniline film 40 is different from the cell culture membrane 10 shown in FIG. 1 in that the polyaniline film 40 does not enter the through hole 23 and closes the first opening portion 23a.

(C5) As shown in “C5” of FIG. 9, a cell culture membrane 510 includes the membrane body 20, the metal film 30, and the polyaniline film 40. Since the membrane body 20 and the metal film 30 have the same structure as those in the cell culture membrane 210 shown in FIG. 8, description thereof will be omitted. The polyaniline film 40 is different from the cell culture membrane 210 shown in FIG. 8 in that the polyaniline film 40 does not enter the through hole 23 and closes the first opening portion 23a.

(C6) As shown in “C6” of FIG. 9, a cell culture membrane 610 includes the membrane body 20, the metal film 30, and the polyaniline film 40. Since the membrane body 20 and the metal film 30 have the same structure as those in the cell culture membrane 310 shown in FIG. 8, description thereof will be omitted. The polyaniline film 40 is different from the cell culture membrane 310 shown in FIG. 8 in that the polyaniline film 40 does not enter the through hole 23 and closes the first opening portion 23a.

As described above, the cell culture membrane 10 shown in FIG. 1 is made hydrophilic by ashing in step S16 of the manufacturing process shown in FIG. 2. On the other hand, in the manufacturing processes of the embodiments (C4) to (C6) shown in FIG. 8, step S16 is not performed. Accordingly, the polyaniline film 40 is formed so as not to enter the through hole 23 or the hole 24 but to close the through hole 23 or the hole 24. Since the polyaniline film 40 is formed on the metal film 30 regardless of whether the polyaniline film 40 has entered the through hole 23 or the hole 24, the pH of the culture solution 305 can be measured.

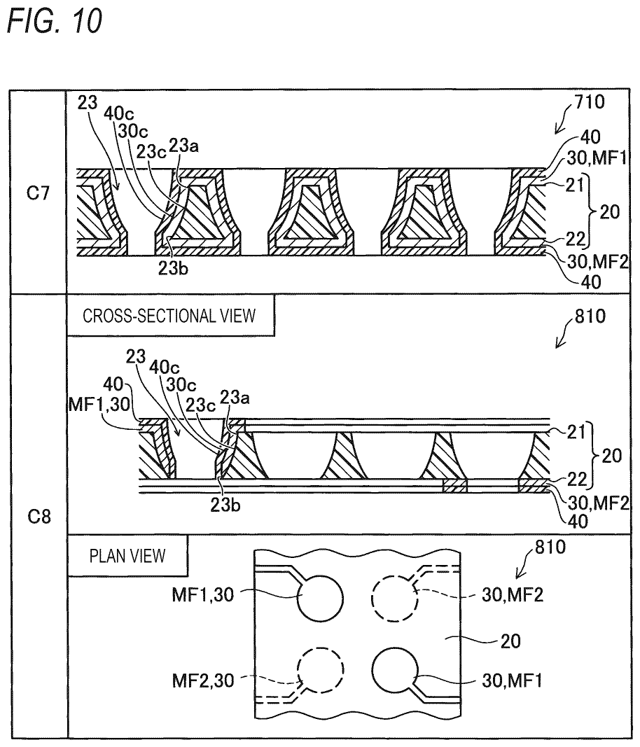

FIG. 10 shows views of embodiments (C7) and (C8), which are other embodiments, of the cell culture membrane 10. The same configurations as those of the above embodiment are denoted by the same reference numerals, and detailed description thereof will be appropriately omitted.

(C7) As shown in “C7” of FIG. 10, a cell culture membrane 710 includes the membrane body 20, the metal film 30, and the polyaniline film 40. The metal film 30 includes the first metal film MF1 and a second metal film MF2. The second metal film MF2 is formed to overlap the second face 22 of the membrane body 20. The cell culture membrane 710 is produced similarly to the manufacturing process shown in FIG. 2. However, in step S14, after the metal film 30 is formed by depositing a metal on the first face 21, after the arrangement is changed such that the second face 22 faces upward on the substrate SB, a metal is deposited on the second face 22 to form the second metal film MF2.

(C8) As shown in “C8” of FIG. 10, a cell culture membrane 810 has the membrane body 20, the metal film 30, and the polyaniline film 40. The metal film 30 includes the first metal film MF1 and the second metal film MF2. The second metal film MF2 is formed to overlap the second face 22 of the membrane body 20. Different from the above embodiment (C7), the first metal film MF1 and the second metal film MF2 are formed in one portion of the membrane body 20. In a “plan view” of “C8” in FIG. 10, the first metal film MF1 is indicated by a solid line, and the second metal film MF2 is indicated by a broken line. The first metal film MF1 and the second metal film MF2 are formed at positions away from each other in a membrane face direction. Accordingly, the first metal film MF1 and the second metal film MF2 are not electrically connected. The cell culture membrane 810 is produced similarly to that in the embodiment (C7). However, in step S14, the metal film 30 is partially formed by being masked such that the metal film 30 is formed in a desired portion. According to this embodiment, when different cells are cultured on the first face 21 and the second face 22 of the cell culture membrane 810, respectively, pH can be measured for each cell.

### D. Another Embodiment of Culture Container

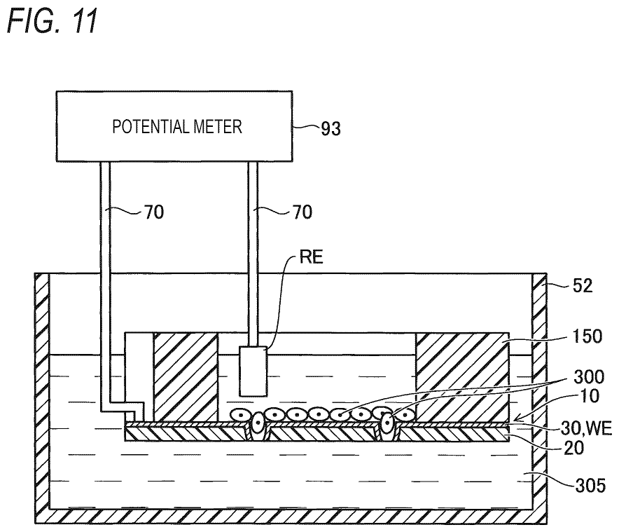

FIG. 11 is a view showing another embodiment of the culture container 80. The same configurations as those of the above embodiment are denoted by the same reference numerals, and detailed description thereof will be appropriately omitted. As shown in FIG. 11, a culture container 180 of the present embodiment is made of a resin and has a cylindrical shape. The cell culture membrane 10 is attached to an end portion of the cylindrical member 50 in the axial direction. In the step of performing culture, the cell culture membrane 10 is immersed in the well 52 into which the culture solution 305 is placed, and the culture is performed.

According to the embodiment described above, the cell culture membrane 10 includes the membrane body 20 having the plurality of through holes 23, the metal film 30, and the polyaniline film 40 formed to overlap the metal film 30. The metal film 30 includes the first metal film MF1. Each of the plurality of through holes 23 is open at least at the first face 21. Accordingly, in a case where the cells 300 are cultured on the polyaniline film 40, the pH in the vicinity of the cells can be accurately measured by using the metal film 30 as an electrode and using the polyaniline film 40 as a response film of protons.

The first metal film MF1 has the metal inner peripheral portion 30c formed to cover the inner peripheral portion 23c. The polyaniline film 40 has the polyaniline inner peripheral portion 40c formed to cover the metal inner peripheral portion 30c. Thus, since it is possible to increase an area of the polyaniline film 40 in contact with or close to the cells 300 that have entered the through holes 23, the measurement accuracy of the pH can be further improved. Further, when the first cell 301 is cultured on the first face 21 and the second cell 302 is cultured on the second face 22, since the first cell 301 and the second cell 302 can be brought into contact with each other through the second opening portion 23b, the cellular condition closer to that in the living body can be created.

The membrane body 20 is made of polyurethane. Accordingly, the membrane body 20 having the plurality of through holes 23 whose first average hole diameter is larger than the second average hole diameter can be easily produced. The second average hole diameter is 7 μm or less. Therefore, when the cells 300 having a size of about 10 μm are cultured, it is possible to suppress the wrap-around of the cells 300.

The measurement method includes step S30 as a first step of culturing the cells 300 on the metal film 30, step S32 as a second step of measuring a potential difference, and step S34 as a third step of determining pH. Accordingly, when the cells 300 are cultured on the cell culture membrane 10, it is possible to measure the pH using the metal film 30 as the electrode.

According to another embodiment (C3), the cell culture membrane 10 has the holes 24 that are a non-through holes. The first metal film MF1 has the metal inner peripheral portion 30f covering the inner peripheral part 23d that defines the hole 24. The polyaniline film 40 has the polyaniline inner peripheral portion 40e covering the metal inner peripheral portion 30f. Thus, since it is possible to increase an area of the polyaniline film 40 in contact with or close to the cells 300 that have entered the holes 24, the measurement accuracy of the pH can be further improved.

According to the other embodiment (C1), the first metal film 30 has the metal closing portions 30d closing the second opening portions 23b. The metal closing portion 30d has the crack 30e. Thus, when the first cell 301 is cultured on the first face 21 and the second cell 302 is cultured on the second face 22, the first cell 301 and the second cell 302 can be brought into contact with each other through the crack 30e, and since the size of the crack 30e is smaller than the size of the second opening portion 23b, it is expected that the cellular condition closer to that in the living body can be created.

### E. Examples

### E-1. Production of Cell Culture Membrane

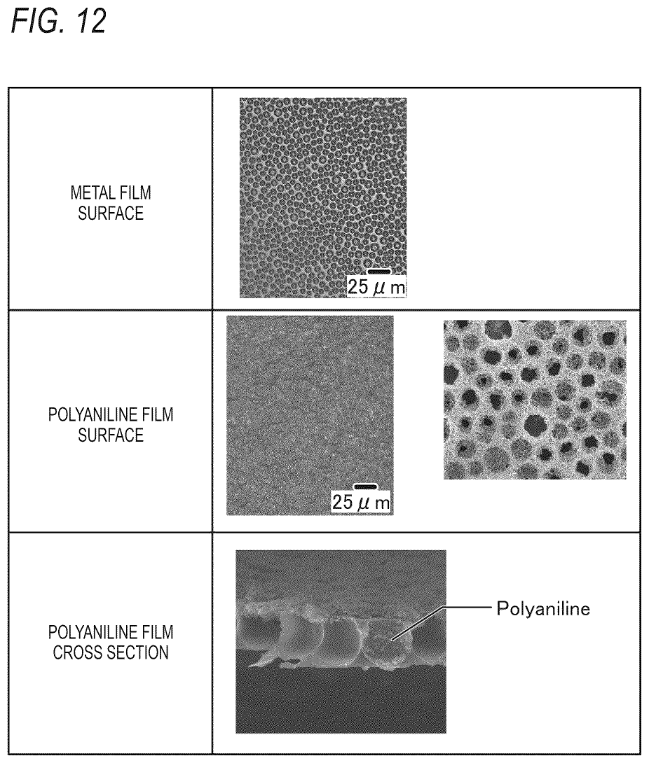

FIG. 12 shows scanning electron microscope (SEM) images of membranes produced in steps of the manufacturing process of the cell culture membrane 10.

A “metal film surface” in FIG. 12 is an SEM image obtained by observing a film face of the film after the formation of the metal film 30 in step S14 shown in FIG. 2. As shown in the “metal film surface” in FIG. 12, the metal film 30 is formed along the inner peripheral portion 23c of the through hole 23.

A “polyaniline film surface” in FIG. 12 is an SEM image obtained by observing a film face of the film after the formation of the polyaniline film 40 in step S18 shown in FIG. 2. A right diagram of the “the polyaniline film surface” in FIG. 12 is an enlarged view of the left diagram. A “polyaniline film cross section” in FIG. 12 is an SEM image obtained by observing a cross section of the film after the formation of the polyaniline film 40 in step S18 shown in FIG. 2.

As shown in the “polyaniline film cross section” in FIG. 12, the polyaniline film 40 is formed along the inner peripheral portion 23c of the through hole 23. As shown in the right diagram of the “polyaniline film surface” in FIG. 12, regarding some of the through holes 23 among all the through holes 23 formed in the membrane body 20, the polyaniline film 40 may be formed to close the opening of the through holes 23 without the polyaniline film 40 entering the through holes 23. Regarding all the through holes 23 formed in the membrane body 20, even when the polyaniline film 40 does not enter the through holes 23, the measurement accuracy of the pH can be improved by the polyaniline film 40 formed to enter the through holes 23. As described above, whether the polyaniline film 40 enters the through hole 23 can be controlled by the presence or absence of the ashing step in step S16.

### E-2. Culture Results

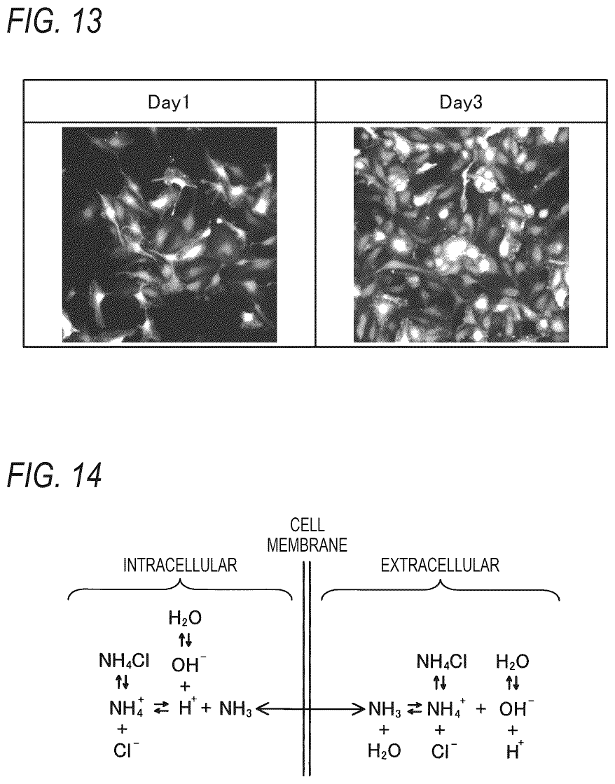

FIG. 13 shows SEM images obtained by observing the cell culture membrane 10 after the cells were cultured on the cell culture membrane 10. A left diagram of FIG. 13 is an SEM image in which the number of days of culture was one day. A right diagram of FIG. 13 is an SEM image in which the number of days of culture was three days. As shown in FIG. 13, the cells 300 could be well cultured using the cell culture membrane 10.

### E-3. Measurement of pH During Cell Culture

FIG. 14 is a diagram illustrating a chemical reaction when ammonium chloride (NH₄Cl) was added to an aqueous solution containing the cells.

As indicated by “extracellular” in FIG. 14, when ammonium chloride is added to the aqueous solution, ammonium ions (NH₄⁺) and hydroxide ions (OH⁻) react with each other to obtain ammonia (NH₃). Accordingly, the hydroxide ions are reduced relative to protons (H⁺), and the aqueous solution becomes acidic.

As shown in FIG. 14, “extracellular” ammonium passes through the cell membrane and moves into “intracellular”. As indicated by “intracellular” of FIG. 14, ammonium reacts with protons to form ammonium ions. Accordingly, the protons are reduced relative to the hydroxide ions, and the inside of the cells becomes alkali.

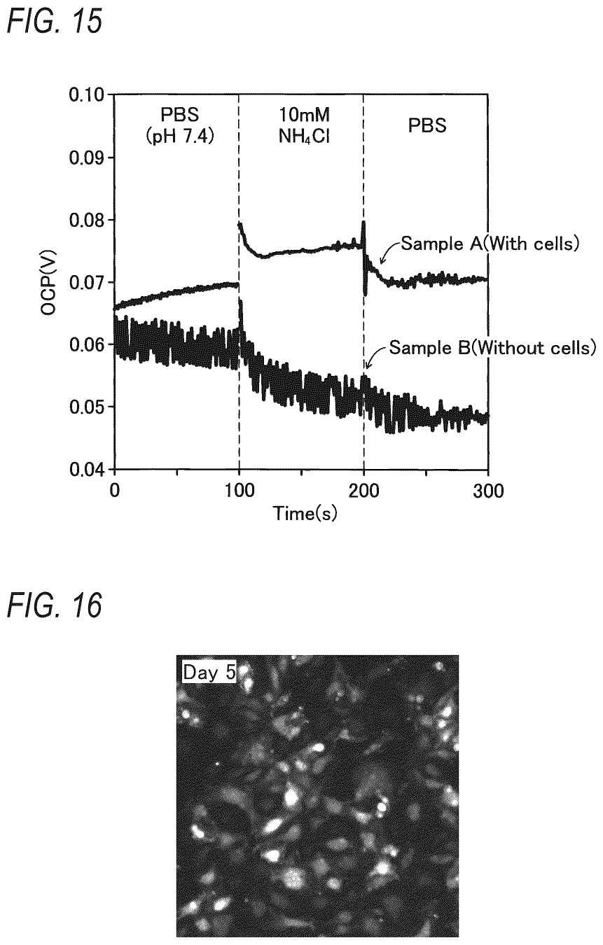

FIG. 15 shows measurement results of the open circuit potential when ammonium chloride was added to phosphate buffered saline (PBS). As shown in FIG. 15, change of the open circuit potential over time when ammonium chloride was added to the culture solution was measured for the cell culture membrane 10 in which (Sample A) cells were cultured and the cell culture membrane 10 in which (Sample B) cells were not cultured. Culture conditions of the used solution and Sample A are as follows.

### Solution

Solution A: PBS (pH 7.4)
Solution B: 10 mM NH₄Cl in PBS
Solution C: PBS

### Culture Conditions

Cell: GFP-HUVEC
Number of days of culture: 5 days

FIG. 16 shows an SEM image obtained by observing the cell culture membrane 10 after the cells were cultured on the cell culture membrane 10 of (Sample A). As shown in FIG. 16, the cells could be well cultured.

As shown in FIG. 15, the cell culture membrane 10 was immersed in PBS having pH of 7.4 for each of (Sample A) and (Sample B), and the open circuit potential was measured for 100 seconds. After 100 seconds from the start of measurement, ammonium chloride was added to a concentration of 10 mM, and the open circuit potential was measured for 100 seconds. After 100 seconds from the start of measurement, the solution in which the cell culture membrane 10 was immersed was replaced with PBS, and the open circuit potential was measured for 100 seconds.

As shown in FIG. 15, in (Sample A), the open circuit potential increased when ammonium chloride was added. Thereafter, when the solution was substituted with PBS, the open circuit potential decreased. According to the cell culture membrane 10, the pH in the vicinity of the cells can be accurately measured.

The present disclosure is not limited to the above-described embodiments, and may be implemented by various configurations without departing from the gist of the present disclosure. For example, technical features in the embodiments corresponding to technical features in aspects described in the summary of the invention can be replaced or combined as appropriate to solve a part or all of the above-described problems or to achieve a part or all of the above-described effects. The technical features may be appropriately deleted unless being described as necessary in the present specification.

### REFERENCE SIGNS LIST

10, 110, 210, 310, 410, 510, 610, 710, 810 cell culture membrane
20 membrane body
21 first face
22 second face
23 through hole
23a first opening portion
23b second opening portion
23c, 23d inner peripheral portion
24 hole
30 metal film
30c, 30f metal inner peripheral portion
30d metal closing portion
30e crack
31 first metal layer
32 second metal layer
33 third metal layer
40 polyaniline film
40c, 40e polyaniline inner peripheral portion
40d polyaniline closing portion
50, 150 cylindrical member
60 resin container
70 conductive wire
80 culture container
90 measurement system
93 potential meter
300 cell
301 first cell
302 second cell
305 culture solution
Da first hole diameter
Db second hole diameter
MF1 first metal film
MF2 second metal film
RE reference electrode
SB substrate
WE working electrode

What is claimed is:

1. A cell culture membrane comprising:
a membrane body having a first face and a second face located on a side opposite to the first face;
a metal film including at least one of a first metal film provided to overlap the first face or a second metal film provided to overlap the second face; and
a polyaniline film provided to overlap the metal film, wherein
the membrane body has a plurality of holes, and each of the plurality of holes has a mortar shape and opens at least at the first face.

2. The cell culture membrane according to claim 1, wherein
each of the plurality of holes is a non-through hole that does not open at the second face and has an inner peripheral portion that defines the each of the plurality of holes,
the metal film does not include the second metal film and includes the first metal film,
the first metal film has a metal inner peripheral portion covering the inner peripheral portion, and
the polyaniline film has a polyaniline inner peripheral portion covering the metal inner peripheral portion.

3. The cell culture membrane according to claim 1, wherein
each of the plurality of holes is a through hole penetrating from the first face to the second face, and has a first opening portion opening at the first face, a second opening portion opening at the second face, and an inner peripheral portion connecting the first opening portion to the second opening portion,
the metal film does not include the second metal film and includes the first metal film,
the first metal film has a metal inner peripheral portion provided to cover the inner peripheral portion, and
the polyaniline film has a polyaniline inner peripheral portion provided to cover the metal inner peripheral portion.

4. The cell culture membrane according to claim 1, wherein
each of the plurality of holes is a through hole penetrating from the first face to the second face, and has a first opening portion opening at the first face, a second opening portion opening at the second face, and an inner peripheral portion connecting the first opening portion to the second opening portion,
the metal film does not include the second metal film and includes the first metal film,
the first metal film has a metal inner peripheral portion provided to cover the inner peripheral portion and a metal closing portion closing the second opening portion,
the polyaniline film has a polyaniline inner peripheral portion provided to cover the metal inner peripheral portion and the metal closing portion, and
the metal closing portion has a crack.

5. The cell culture membrane according to claim 3, wherein
the plurality of holes have a first average hole diameter at the first face larger than a second average hole diameter at the second face.

6. The cell culture membrane according to claim 4, wherein
the plurality of holes have a first average hole diameter at the first face larger than a second average hole diameter at the second face.

7. The cell culture membrane according to claim 1, wherein
the membrane body comprises polyurethane.

8. The cell culture membrane according to claim 2, wherein
the membrane body comprises polyurethane.

9. The cell culture membrane according to claim 3, wherein
the membrane body comprises polyurethane.

10. The cell culture membrane according to claim 4, wherein
the membrane body comprises polyurethane.

11. The cell culture membrane according to claim 5, wherein
the second average hole diameter is 7 μm or less.

12. The cell culture membrane according to claim 6, wherein
the second average hole diameter is 7 μm or less.

13. The cell culture membrane according to claim 1, wherein
the metal film includes a first metal layer mainly made of gold.

14. The cell culture membrane according to claim 13, wherein
the metal film further includes a second metal layer mainly made of platinum and a third metal layer mainly made of titanium.

15. A measurement method of pH comprising:
immersing the cell culture membrane according to claim 1 in a culture solution to culture a cell on the polyaniline film of the cell culture membrane;
inserting a reference electrode into the culture solution, using the metal film as a working electrode, and measuring an electrical potential difference between the reference electrode and the working electrode; and
determining pH of the culture solution using the measured electrical potential difference.
